(19) United States
(12) Patent Application Publication
(10) Pub. No.: US 2006/0100269 A1
(43) Pub. Date: May 11, 2006

(54) Use of 10-aminoaliphatyl-dibenz[b,f]oxepines for the treatment of degenerative ocular disorders

(76) Inventors: George N Lambrou, Strasbourg (FR); Elisabeth Latour, Bartenheim-La Chaussee (FR); Peter Waldmeier, Ettingen (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/543,361

(22) PCT Filed: Jan. 28, 2004
(86) PCT No.: PCT/EP04/00746

§ 371 Date: Jul. 26, 2005

(30) Foreign Application Priority Data
Jan. 29, 2003 (GB) 0302091.4
Feb. 3, 2003 (GB) 0302436.1
Oct. 10, 2003 (GB) 0323829.2

(51) Int. Cl. A61K 31/335

(52) U.S. Cl. 514/450

(57) ABSTRACT

The present invention relates to novel uses of 10-aminoaliphatyl-dibenz[b,f]oxepines for the treatment of degenerative ocular disorders.

The invention relates to novel uses of 10-aminoaliphatyl-dibenz[b,f]oxepines of formula I 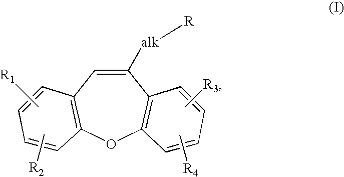
wherein alk is a divalent aliphatic radical,
R is an amino group that is unsubstituted or mono- or di-substituted by monovalent aliphatic and/or araliphatic radicals or disubstituted by divalent aliphatic radicals, and
R₁, R₂, R₃ and R₄ are each, independently of the others, hydrogen, lower alkyl, lower alkoxy, halogen or trifluoromethyl,
and pharmaceutically acceptable salts thereof, for the treatment of ocular diseases and in the preparation thereof.

Certain compounds of formula I are decribed in German Offenlegungsschrift No. 1 793 521 as adrenolytic and central nervous system-dampening active ingredients of medicaments.

Certain compounds of formula I are described in EP726265 to be useful in the treatment of neurodegenerative disorders, especially those in which apoptotic necrocytosis plays a part, such as cerebral Ischaemias, Aizheimer's and Parkinson's disease, amyotrophic lateral sclerosis, glaucoma and also general or diabetic peripheral neuropathies.

The present Invention relates to a novel use of the compounds In accordance to the above formula I In the manufacture of a medicament for the treatment of a degenerative ocular disorder.

Degenerative ocular disorders which may be treated according to this Invention include an ocular disease and disorder which may directly or indirectly involve the degeneration of retinal or corneal cells, in particular by apoptosis, including ischemic retinopathies in general, anterior ischemic optic neuropathy, all forms of optic neuritis, age-related macular degeneration (AMD), in its dry forms (dry AMD) and wet forms (wet AMD), diabetic retinopathy, cystoid macular edema (CME), retinal detachment, retinitis pigmentosa, Stargardt's disease, Best's vitelliform retinal degeneration, Leber's congenital amaurosis and other hereditary retinal degenerations, pathologic myopia, retinopathy of prematurity, and Leber's hereditary optic neuropathy, the after effects of corneal transplantation or of refractive corneal surgery, and herpes keratitis.

Preferably, said ocular disorders are selected from:

Dry AMD, wet AMD, diabetic retinopathy, cystoid macular edema (CME), retinal detachment, pathologic myopia, Leber's hereditary optic neuropathy, retinitis pigmentosa, and other hereditary retinal degenerations, and even more preferably, said ocular disorders are selected from:

Dry AMD, wet AMD and retinal detachment.

In the present description the terms “treatment” or “treat” refer to both prophylactic or preventive treatment as well as curative or disease-modifying treatment, including treatment of patients at risk of contracting the disease or suspected to have contracted the disease as well as patients who are ill or have been diagnosed as suffering from a disease or medical condition.

As used herein, monovalent aliphatic radicals are, for example, lower alkyl, lower alkenyl or lower alkynyl groups that are unsubstituted or substituted by free or etherified or esterified hydroxy or by unsubstituted or aliphatically substituted amino, such as lower alkyl, hydroxy-lower alkyl, lower alkoxy-lower alkyl, lower alkanoyloxy-lower alkyl, lower alkylamino-lower alkyl, di-lower alkylamino-lower alkyl, lower alkyleneamino-lower alkyl, lower alkenyl, hydroxy-lower alkenyl, lower alkoxy-lower alkenyl, lower alkanoyloxy-lower alkenyl, di-lower alkylamino-lower alkenyl, lower alkynyl, hydroxy-lower alkynyl, lower alkoxy-lower alkynyl, lower alkanoyloxy-lower alkynyl or di-lower alkylamino-lower alkynyl. Araliphatic radicals are, for example, phenyl-lower alkyl radicals that are unsubstituted or substituted by lower alkyl, lower alkoxy, halogen and/or by trifluoromethyl.

Amino groups that are mono- or di-substituted by monovalent aliphatic or araliphatic radicals are therefore, for example, lower alkylamino; phenyl-lower alkylamino or phenyl-lower alkyl-lower alkylamino each of which is unsubstituted or substituted by lower alkyl, lower alkoxy, halogen and/or by trifluoromethyl; hydroxy-lower alkylamino, lower alkoxy-lower alkylamino, lower alkanoyloxy-lower alkylamino, lower alkylamino-lower alkylamino, di-lower alkylamino-lower alkylamino, lower alkyleneamino-lower alkylamino, lower alkenylamino, hydroxy-lower alkenylamino, lower alkoxy-lower alkenylamino, lower alkanoyloxy-lower alkenylamino, di-lower alkylamino-lower alkenylamino, lower alkynylamino, hydroxy-lower alkynylamino, lower alkoxy-lower alkynylamino, lower alkanoyloxy-lower alkynylamino, di-lower alkylamino-lower alkynylamino, di-lower alkylamino, di(hydroxy-lower alkyl)amino, hydroxy-lower alkyl-lower alkylamino, di(lower alkoxy-lower alkyl)amino, lower alkoxy-lower alkyl-lower alkylamino, lower alkanoyloxy-lower alkylamino, lower alkanoyloxy-lower alkyl-lower alkylamino, di-lower alkylamino-lower alkylamino, di-lower alkylamino-lower alkyl-lower alkylamino, di-lower alkenylamino, lower alkenyl-lower alkylamino, hydroxy-lower alkenyl-lower alkylamino, di(lower alkoxy-lower alkenyl)amino, lower alkoxy-lower alkenyl-lower alkylamino, lower alkanoyloxy-lower alkenyl-lower alkylamino, di-lower alkylamino-lower alkenyl-lower alkylamino, lower alkynyl-lower alkylamino, lower alkoxy-lower alkynyl-lower alkylamino, lower alkanoyloxy-lower alkynyl-lower alkylamino or di-lower alkylamino-lower alkynyl-lower alkylamino.

Divalent aliphatic radicals are, for example, lower alkylene radicals and, as a component of an amino group disubstituted by a divalent aliphatic radical, also aza-, oxa- or thia-lower alkylene radicals, such as 3- or 4-aza-lower alkylene that is unsubstituted or N-substituted by lower alkyl, hydroxy-lower alkyl, lower alkoxy-lower alkyl or by lower alkanoyl, 3- or 4-oxa-lower alkylene or optionally S-oxidised 3- or 4-thia-lower alkylene.

Amino groups disubstituted by divalent aliphatic radicals are, for example, 3- to 8-membered lower alkyleneamino, 3- or 4-aza-lower alkyleneamino that is unsubstituted or N-substituted by lower alkyl, hydroxy-lower alkyl, lower alkoxy-lower alkyl or by lower alkanoyl, 3- or 4-oxa-lower alkyleneamino or optionally S-oxidised 3- or 4-thia-lower alkyleneamino, such as, especially, pyrrolidino, piperidino, di-lower alkylpiperidino, hexamethyleneimino, heptamethyleneimino, piperazino, N′-lower alkylpiperazino, N′-hydroxy-lower alkylpiperazino, N′-lower alkoxy-lower alkylpiperazino, N′-lower alkanoylpiperazino, morpholino, thiomorpholino, S-oxothiomorpholino or S,S-dioxothiomorpholino.

As used herein lower denotes radicals having typically up to and including 7, preferably up to and including 4 carbon atoms.

Di(hydroxy-lower alkyl)amino is, for example, N,N-di(hydroxy-C₂-C₄alkyl)amino, such as N,N-di(2-hydroxyethyl)amino or N,N-di(3-hydroxypropyl)amino.

Di(lower alkoxy-lower alkenyl)amino is, for example, N,N-di(C₁-C₄alkoxy-C₂-C₄alkenyl)amino, such as N,N-di(4-methoxy-but-2-enyl)amino.

Di(lower alkoxy-lower alkyl)amino is, for example, N,N-di(C₁-C₄alkoxy-C₁-C₄alkyl)amino, such as N,N-di(2-methoxyethyl)amino, N,N-di(2-ethoxyethyl)amino or N,N-di(3-methoxypropyl)-amino.

Di-lower alkenylamino is, for example, N,N-di-C₂-C₄alkenylamino, such as N,N-diallylamino or N-methallyl-N-allylamino.

Di-lower alkylamino is, for example, N,N-di-C₁-C₄alkylamino, such as dimethylamino, diethylamino, ethylmethylamino, dipropylamino, methylpropylamino, ethylpropylamino, dibutylamino or butylmethylamino.

Di-lower alkylamino-lower alkenyl-lower alkylamino is, for example, N-(di-C₁-C₄alkylamino-C₂-C₄alkenyl)-N—C₁-C₄alkylamino, such as N-(4-dimethylaminobut-2-enyl)-N-methylamino.

Di-lower alkylamino-lower alkenylamino is, for example, N-(di-C₁-C₄alkylamino-C₂-C₄-alkenyl)amino, such as N-(4-dimethylaminobut-2-enyl)amino.

Di-lower alkylamino-lower alkynylamino is, for example, N-(di-C₁-C₄alkylamino-C₂-C₄-alkynyl)amino, such as N-(4-dimethylaminobut-2-ynyl)amino.

Di-lower alkylamino-lower alkyl-lower alkylamino is, for example, N-(di-C₁-C₄alkylamino-C₂-C₄alkyl)-N—C₁-C₄alkylamino, such as N-(2-dimethylaminoethyl)-N-methylamino, N-(2-dimethylaminoethyl)-N-ethylamino, N-(3-dimethylaminopropyl)-N-methylamino or N-(4-dimethylaminobutyl)-N-methylamino.

Di-lower alkylamino-lower alkylamino is, for example, N-(di-C₄-C₄alkylamino-C₂-C₄alkyl)-amino, such as N-(2-dimethylaminoethyl)amino, N-(2-dlmethylaminoethyl)amino, N-(3-dimethylaminopropyl)amino or N-(4-dimethylaminobutyl)amino.

Halogen is, for example, fluorine, chlorine, bromine and iodine, preferably fluorine, chlorine, and bromine, more preferably chlorine and bromine, even more preferably chlorine.

Hydroxy-lower alkenyl-lower alkylamino Is, for example, N-hydroxy-C₂-C₄alkenyl)-N-(C₁-C₄alkylamino, such as N-(4-hydroxy-but-2-enyl)-N-methylamino.

Hydroxy-lower alkenylamino is, for example, hydroxy-C₂-C₄alkenylamino, such as 4-hydroxy-but-2-ynylamino.

Hydroxy-lower alkynylamino is, for example, hydroxy-C₂-C₄alkynylamino, such as 4-hydroxy-but-2-ynylamino.

Hydroxy-lower alkyl-lower alkylamino is, for example, N-(hydroxy-C₂-C₄alkyl)-N—C₁-C₄alkyl-amino, such as N-(2-hydroxyethyl)-N-methylamino, N-(3-hydroxypropyl)-N-methylamino or N-(4-hydroxybutyl)-N-methylamino.

Hydroxy-lower alkylamino is, for example, hydroxy-C₂-C₄alkylamino, such as 2-hydroxyethyl-amino, 3-hydroxypropylamino or 4-hydroxybutylamino.

N′-Hydroxy-lower alkylpiperazino is, for example, N′-(hydroxy-C₁-C₄alkyl)piperazino, such as

N′-(2-hydroxyethyl)piperazino or N′-(3-hydroxypropyl)piperazino.

N′-Lower alkanoylpiperazino is, for example, N′-C₁-C₇alkanoylpiperazino, such as N′-acetyl-piperazino.

N′-Lower alkoxy-lower alkylpiperazino is, for example, N′-(C₁-C₄alkoxy-C₁-C₄alkyl)piperazino, such as N′-(2-methoxyethyl)piperazino or N′-(3-methoxypropyl)piperazino.

N′-Lower alkylpiperazino is, for example, N′-C₁-C₄alkylpiperazino, such as N′-methyl-piperazino, N′-ethylpiperazino, N′-propylpiperazino or N′-butylpiperazino.

Lower alkoxy is, for example, C₁-C₇alkoxy, preferably C₁-C₅alkoxy, such as methoxy, ethoxy, propyloxy, isopropyloxy, butyloxy, isobutyloxy, sec-butyloxy, tert-butyloxy, pentyloxy or a hexyloxy or heptyloxy group.

Lower alkanoyloxy-lower alkenyl-lower alkylamino is, for example, N—(C₁-C₇alkanoyloxy-C₂-C₄alkenyl)-N-(C₁-C₄alkyl)amino, such as N-(4-acetoxybut-2-enyl)-N-methylamino.

Lower alkanoyloxy-lower alkenylamino is, for example, N—(C₁-C₇alkanoyloxy-C₂-C₄-alkenyl)-amino, such as N-(4-acetoxybut-2-enyl)amino.

Lower alkanoyloxy-lower alkynyl-lower alkylamino is, for example, N—(C₁-C₇alkanoyloxy-C₂-C₄alkynyl)-N-(C₁-C₄alkyl)amino, such as N-(4-acetoxybut-2-ynyl)-N-methylamino.

Lower alkanoyloxy-lower alkynylamino is, for example, N—(C₁-C₇alkanoyloxy-C₂-C₄-alkynyl)-amino, such as N-(4-acetoxybut-2-ynyl)amino.

Lower alkanoyloxy-lower alkyl-lower alkylamino is, for example, N—(C₁-C₇alkanoyloxy-C₂-C₄-alkyl)-N-(C₁-C₄alkyl)amino, such as N-(2-acetoxyethyl)-N-methylamino, N-(2-acetoxyethyl)-N-ethylamino, N-(3-acetoxypropyl)-N-methylamino or N-(4-acetoxybutyl)-N-methylamino.

Lower alkanoyloxy-lower alkylamino is, for example, N—(C₁-C₇alkanoyloxy-C₂-C₄alkyl)amino, such as N-(2-acetoxyethyl)amino, N-(3-acetoxypropyl)amino or N-(4-acetoxybutyl)amino.

Lower alkenyl-lower alkylamino is, for example, N—(C₂-C₇alkenyl)-N-(C₂-C₇alkyl)amino, especially N—(C₂-C₄alkenyl)-N-(C₁-C₄alkyl)amino, such as N-vinyl-N-methylamino, N-allyl-N-methylamino, N-allyl-N-ethylamino, N-but-2-enyl-N-methylamino or N-but-3-enyl-N-methyl amino.

Lower alkenylamino is, for example, N—(C₂-C₇alkenyl)amino, especially N—(C₂-C₄-alkenyl)-amino, such as vinylamino, allylamino, but-2-enylamino or N-but-3-enylamino, especially allylamino.

Lower alkynyl-lower alkylamino is, for example, N—(C₂-C₄alkynyl)-N—(C₁-C₄alkyl)amino, such as N-propargyl-N-methylamino, N-but-2-ynyl-N-methylamino or N-but-3-ynyl-N-methylamino.

Lower alkynylamino is, for example, N—(C₂-C₇alkynyl)amino, especially N—(C₂-C₄alkynyl)-amino, such as propargylamino, but-2-ynylamino or N-but-3-ynylamino, especially propargyl-amino.

Lower alkoxy is, for example, C₁-C₇alkoxy, preferably C₁-C₄alkoxy, such as methoxy, ethoxy, propyloxy, isopropyloxy or butyloxy, but may also be isobutyloxy, sec-butyloxy, tert-butyloxy or a C₅-C₇alkoxy group, such as a pentyloxy, hexyloxy or heptyloxy group.

Lower alkoxy-lower alkenyl-lower alkylamino is, for example, N—(C₁-C₄alkoxy-C₂-C₄alkenyl)-N-(C₁-C₄alkyl)amino, such as N-(4-methoxybut-2-enyl)-N-methylamino, N-(4-methoxybut-2-enyl)-N-ethylamino or N-(4-ethoxybut-2-enyl)-N-methylamino.

Lower alkoxy-lower alkenylamino is, for example, N—(C₁-C₄alkoxy-C₂-C₄alkenyl)amino, such as N-(4-methoxybut-2-enyl)amino or N-(4-ethoxybut-2-enyl)amino.

Lower alkoxy-lower alkynyl-lower alkylamino is, for example, N—(C₁-C₄alkoxy-C₂-C₄alkynyl)-N-(C₁-C₄alkyl)amino, such as N-(4-methoxybut-2-ynyl)-N-methylamino, N-(4-methoxybut-2-ynyl)-N-ethylamino or N-(4-ethoxybut-2-ynyl)-N-methylamino.

Lower alkoxy-lower alkynylamino is, for example, N—(C₁-C₄alkoxy-C₂-C₄alkynyl)amino, such as N-(4-methoxybut-2-ynyl)amino, N-(4-ethoxybut-2-ynyl)amino or N-(4-propyloxybut-2-ynyl)-amino.

Lower alkoxy-lower alkylamino is, for example, C₁-C₄alkoxy-C₂-C₄alkylamino, such as 2-methoxyethylamino, 2-ethoxyethylamino, 2-propyloxyethylamino, 3-methoxypropylamino, 3-ethoxypropylamino, 4-methoxybutylamino, 2-isopropyloxyethyl amino or 2-butyloxyethylamino.

Lower alkoxy-lower alkyl-lower alkylamino is, for example, N—(C₁-C₄alkoxy-C₂-C₄alkyl)-N-(C₁-C₄alkyl)amino, such as N-(2-methoxyethyl)-N-methylamino, N-(2-ethoxyethyl)-N-methyl-amino, N-(2-propyloxyethyl)-N-methylamino, N-(3-methoxypropyl)-N-methylamino, 3-ethoxy-propylamino or N-(4-methoxybutyl)-N-methylamino.

Lower alkyl is, for example, C₁-C₇alkyl, preferably C₁-C₄alkyl, such as methyl, ethyl, propyl, isopropyl or butyl, but may also be isobutyl, sec-butyl, tert-butyl or a C₅-C₇alkyl group, such as a pentyl, hexyl or heptyl group.

Lower alkylamino is, for example, C₁-C₇alkylamino, preferably C₁-C₄alkylamino, such as methylamino, ethylamino, propylamino, isopropylamino or butylamino, but may also be isobutylamino, sec-butylamino or tert-butylamino or a C₅-C₇alkylamino group, such as a pentylamino, hexylamino or heptylamino group, and is especially methylamino or propyl-amino.

Lower alkylamino-lower alkylamino is, for example, N—(C₁-C₄alkylamino-C₂-C₄alkyl)amino, such as N-(2-methylaminoethyl)amino, N-(3-methylaminopropyl)amino, N-(4-methylamino-butyl)amino, N-(2-ethylaminoethyl)amino, N-(3-ethylaminopropyl)amino or N-(4-ethylamino-butyl)amino.

Lower alkyleneamino-lower alkylamino is, for example, 3- to 8-membered alkyleneamino-C₂-C₄alkylamino, such as 2-pyrrolidinoethylamino, 2-piperidinoethylamino, 2-dimethyl-piperidinoethylamino, 2-hexamethylenelminoethylamino, 3-pyrrolidinopropylamino, 3-piperidinopropylamino, 3-dimethylpiperidinopropylamino or 3-hexamethyleneiminopropyl-amino.

Phenyl-lower alkyl-lower alkylamino is, for example, N-(phenyl-C₁-C₄alkyl)-N-(C₁-C₄alkyl)-amino, such as N-benzyl-N-methylamino, N-(2-phenylethyl)-N-methylamino or N-(4-phenyl-butyl)-N-methylamino.

Phenyl-lower alkylamino is, for example, phenyl-C₁-C₄alkylamino, such as benzylamino, 1- or 2-phenylethylamino, 3-phenylpropylamino or 4-phenylbutylamino.

Salts of compounds of formula I are, for example, the pharmaceutically acceptable acid addition salts thereof with suitable mineral acids, such as hydrohalic acids, sulfuric acid or phosphoric acid, for example hydrochlorides, hydrobromides, sulfates, hydrogen sulfates or phosphates, or salts with suitable allphatic or aromatic sulfonic acids or N-substituted sulfamic acids, for example methanesulfonates, benzenesulfonates, p-toluenesulfonates or N-cyclohexylsulfamates (cyclamates), or salts of suitable organic carbonic acids, such as benzoic add, fumaric acid and maleic acid. A preferred acid addition salt is for example hydrogen fumarate or hydrogen maleate.

Certain compounds of formula I are preferred in the use or the method of the main independent and dependant claims of the present invention. Accordingly, preferred are compounds of formula I wherein
alk is lower alkylene,
R is amino, lower alkylamino; phenyl-lower alkylamino or phenyl-lower alkyl-lower alkylamino each of which is unsubstituted or substituted by lower alkyl, lower alkoxy, halogen and/or by trifluoromethyl; hydroxy-lower alkylamino, lower alkoxy-lower alkylamino, lower alkanoyloxy-lower alkylamino, lower alkylamino-lower alkylamino, di-lower alkylamino-lower alkylamino, lower alkyleneamino-lower alkyl amino, lower alkenylamino, hydroxy-lower alkenylamino, lower alkoxy-lower alkenylamino, lower alkanoyloxy-lower alkenylamino, di-lower alkylamino-lower alkenylamino, lower alkynylamino, hydroxy-lower alkynylamino, lower alkoxy-lower alkynylamino, lower alkanoyloxy-lower alkynylamino, di-lower alkylamino-lower alkynylamino, di-lower alkylamino, di(hydroxy-lower alkyl)amino, hydroxy-lower alkyl-lower alkylamino, di(lower alkoxy-lower alkyl)amino, lower alkoxy-lower alkyl-lower alkylamino, lower alkanoyloxy-lower alkylamino, lower alkanoyloxy-lower alkyl-lower alkylamino, di-lower alkylamino-lower alkylamino, di-lower alkylamino-lower alkyl-lower alkylamino, di-lower alkenylamino, lower alkenyl-lower alkylamino, hydroxy-lower alkenyl-lower alkylamino, di(lower alkoxy-lower alkenyl)amino, lower alkoxy-lower alkenyl-lower alkylamino, lower alkanoyloxy-lower alkenyl-lower alkylamino, di-lower alkylamino-lower alkenyl-lower alkyl-amino, lower alkynyl-lower alkylamino, lower alkoxy-lower alkynyl-lower alkylamino, lower alkanoyloxy-lower alkynyl-lower alkylamino, di-lower alkylamino-lower alkynyl-lower alkyl-amino, 3- to 8-membered lower alkyleneamino; 3- or 4-aza-lower alkyleneamino that is unsubstituted or N-substituted by lower alkyl, hydroxy-lower alkyl, lower alkoxy-lower alkyl or by lower alkanoyl; 3- or 4-oxa-lower alkyleneamino or optionally S-oxidised 3- or 4-thia-lower alkyleneamino and
R₁, R₂, R₃ and R₄ are each, independently of the others, hydrogen, lower alkyl, lower alkoxy, halogen or trifluoromethyl,
   and pharmaceutically acceptable salts thereof, and also to novel compounds as defined above of formula I as such, especially those in which R is other than amino that is unsubstituted or mono- or di-substituted by lower alkyl, pyrrolidino, piperidino, morpholino, N′-methylpiperazino, N′-(2-hydroxyethyl)piperazino, N′-(2-acetoxyethyl)piperazino, N′-(2-pivaloyloxyethyl)piperazino and N′-methylhomopiperazino when alk is methylene or ethylidene and R₁, R₂, R₃ and R₄ are hydrogen, lower alkyl, lower alkoxy and/or halogen, and to salts thereof.

Also preferred are compounds of formula I wherein
alk is lower alkylene,
R is amino, lower alkylamino; phenyl-lower alkylamino or phenyl-lower alkyl-lower alkylamino each of which is unsubstituted or substituted by lower alkyl, lower alkoxy, halogen and/or by trifluoromethyl; lower alkenylamino, lower alkynylamino, di-lower alkylamino, 3- to 8-membered lower alkyleneamino; 3- or 4-aza-lower alkyleneamino that is unsubstituted or N-substituted by lower alkyl, hydroxy-lower alkyl, lower alkoxy-lower alkyl or by lower alkanoyl; 3- or 4-oxa-lower alkyleneamino or optionally S-oxidised 3- or 4-thia-lower alkyleneamino and R₁, R₂, R₃ and R₄ are each, independently of the others, hydrogen, lower alkyl, lower alkoxy, halogen or trifluoromethyl,
   and pharmaceutically acceptable salts thereof, and also novel compounds as defined above of formula I as such, especially those in which R is other than amino that is unsubstituted or mono- or di-substituted by lower alkyl, pyrrolidino, piperidino, morpholino, N′-methylpiperazino, N′-(2-hydroxyethyl)piperazino, N′-(2-acetoxyethyl)piperazino, N′-(2-pivaloyloxyethyl)piperazino and N′-methylhomopiperazino when alk is methylene or ethylidene and R₁, R₂, R₃ and R₄ are hydrogen, lower alkyl, lower alkoxy and/or halogen, and to salts thereof.

More preferred are compounds of formula I wherein
alk is methylene,
R is amino, C₁-C₄alkylamino, such as methylamino, ethylamino, propylamino or butylamino; phenyl-C₁-C₄alkylamino, such as benzylamino or phenethylamino, that is unsubstituted or substituted by C₁-C₄alkyl, such as methyl, C₁-C₄alkoxy, such as methoxy, halogen having an atomic number of up to and including 35, such as chlorine or bromine, and/or by trifluoromethyl; phenyl-C₁-C₄alkyl-C₁-C₄alkylamino, such as N-benzyl-N-methylamino, that is unsubstituted or substituted by C₁-C₄alkyl, such as methyl, C₁-C₄alkoxy, such as methoxy, halogen having an atomic number of up to and including 35, such as chlorine or bromine, and/or by trifluoromethyl; C₂-C₇alkenylamino, such as allylamino, methallylamino or but-2-enylamino, C₂-C₇alkynylamino, such as propargylamino or but-2-ynylamino, N—C₂-C₇alkenyl-N—C₁-C₄alkylamino, such as N-allyl-N-methylamino, N-allyl-N-ethylamino, N-methallyl-N-methylamino or N-but-2-enyl-N-methylamino, N—C₂-C₇alkynyl-N—C₁-C₄alkylamino, such as N-propargyl-N-methylamino, N-propargyl-N-ethylamino or N-but-2-ynyl-N-methylamino, di-C₁-C₄alkylamino, such as dimethylamino, diethylamino, N-methyl-N-propylamino or N-butyl-N-methylamino, pyrrolidino, piperidino, morpholino, piperazino, N′-C₁-C₄alkylpiperazino, such as N′-methylpiperazino, or N′-(hydroxy-C₂-C₄alkyl)piperazino, such as N′-(2-hydroxyethyl)-piperazino, and
R₁, R₂, R₃ and R₄ are each, independently of the others, hydrogen, C₁-C₄alkyl, such as methyl, C₁-C₄alkoxy, such as methoxy, halogen such as chlorine or bromine, or trifluoromethyl,
   and pharmaceutically acceptable salts thereof, and also to novel compounds as defined above of formula I as such, especially those in which R is other than amino, C₁-C₄alkylamino, di-C₁-C₄alkylamino, pyrrolidino, piperidino, morpholino and N′-methylpiperazino when R₁, R₂, R₃ and R₄ are hydrogen, C₁-C₄alkyl, C₁-C₄alkoxy and/or halogen, and to salts thereof.

Especially preferred are compounds of formula I wherein
alk is methylene,
R is C₂-C₇alkenylamino, such as allylamino, methallylamino or but-2-enylamino, C₂-C₇alkynylamino, such as propargylamino or but-2-ynylamino, N—C₂-C₇alkenyl-N—C₁-C₄alkyl-amino, such as N-allyl-N-methylamino, N-allyl-N-ethylamino, N-methallyl-N-methylamino or N-but-2-enyl-N-methylamino, N—C₂-C₇alkynyl-N—C₁-C₄alkylamino, such as N-propargyl-N-methylamino, N-propargyl-N-ethylamino or N-but-2-ynyl-N-methylamino, or pPyrrolidino, piperidino or morpholino,
R₁ and R₃ are each, independently of the others, hydrogen, C₁-C₄alkyl, such as methyl, C₁-C₄alkoxy, such as methoxy, halogen such as chlorine or bromine, or trifluoromethyl, and R₂ and R₄ are hydrogen,
   and to salts thereof.

Especially preferred are compounds of formula I wherein alk is methylene,
R is C₂-C₇alkenylamino, such as allylamino, methallylamino or but-2-enylamino, C₂-C₇alkynylamino, such as propargylamino or but-2-ynylamino, N—C₂-C₇alkenyl-N—C₁-C₄alkyl-amino, such as N-allyl-N-methylamino, N-allyl-N-ethylamino, N-methallyl-N-methylamino or N-but-2-enyl-N-methylamino, N—C₂-C₇alkynyl-N—C₁-C₄alkylamino, such as N-propargyl-N-methylamino, N-propargyl-N-ethylamino or N-but-2-ynyl-N-methylamino, or phenyl-C₁-C₄alkylamino, such as benzylamino or phenethylamino, that is unsubstituted or substituted by C₁-C₄alkyl, such as methyl, C₁-C₄alkoxy, such as methoxy, halogen such as chlorine or bromine, and/or by trifluoromethyl, and
R₁, R₂, R₃ and R₄ are hydrogen,
   and to salts thereof.

Most preferred are compounds of formula I wherein
alk is methylene,
R is C₂-C₇alkynylamino, such as propargylamino or but-2-ynylamino, N—C₂-C₇alkynyl-N-C₁-C₄alkylamino, such as N-propargyl-N-methylamino, N-propargyl-N-ethylamino or N-but-2-ynyl-N-methylamino, and
R₁, R₂, R₃ and R₄ are hydrogen,
   and to salts thereof.

The invention relates specifically to the use of
N-(dibenz[b,f]oxepin-10-ylmethyl)-N-methyl-N-prop-2-ynylamine;
N-allyl-N-(dibenz[b,f]oxepin-10-ylmethyl)amine;
N-allyl-N-(dibenz[b,f]oxepin-10-ylmethyl)-N-methylamine;
N-(dibenz[b,f]oxepin-10-ylmethyl)amine;
N-(dibenz[b,f]oxepin-10-ylmethyl)-N-prop-2-ynylamine;
N-(dibenz[b,f]oxepin-10-ylmethyl)-N-propylamine;
N-(dibenz[b,f]oxepin-10-ylmethyl)-N-methyl-N-propylamine;
1-dibenz[b,f]oxepin-10-ylmethyl-piperidine;
4-dibenz[b,f]oxepin-10-ylmethyl-morpholine;
N-(1-chloro-dibenz[b,f]oxepin-10-ylmethyl)-N-methyl-N-prop-2-ynyl amine;
1-(1-chloro-dibenz[b,f]oxepin-10-ylmethyl)-pyrrolidine;
N-(1-chloro-dibenz[b,f]oxepin-10-ylmethyl)-N-methyl-N-propylamine;
N-methyl-N-prop-2-ynyl-N-(3-trifluoromethyl-dibenz[b,f]oxepin-10-ylmethyl)amine
1-(3-trifluoromethyl-dibenz[b,f]oxepin-10-ylmethyl)-pyrrolidine;
N-(7-chloro-dibenz[b,f]oxepin-10-ylmethyl)-N-methyl-N-prop-2-ynylamine;
1-(7-chloro-dibenz[b,f]oxepin-10-ylmethyl)-pyrrolidine;
N-(8-methoxy-dibenz[b,f]oxepin-10-ylmethyl)-N-methyl-N-prop-2-ynylamine;
N-(8-tert-butyl-dibenz[b,f]oxepin-10-ylmethyl)-N-methyl-N-prop-2-ynylamine;
1-(8-tert-butyl-dibenz[b,f]oxepin-10-ylmethyl)-pyrrolidine;
N-(6-chloro-dibenz[b,f]oxepin-10-ylmethyl)-N-methyl-N-prop-2-ynylamine;
1-(6-chloro-dibenz[b,f]oxepin-10-ylmethyl)pyrrolidine;
N-(1-fluoro-dibenz[b,f]oxepin-10-ylmethyl)-N-methyl-N-prop-2-ynylamine;
1-(1-fluoro-dibenz[b,f]oxepin-10-ylmethyl)pyrrolidine;
N-(dibenz[b,f]oxepin-10-ylmethyl)-N-benzylamine;
N-benzyl-N-(dibenz[b,f]oxepin-10-ylmethyl)-N-methylamine;
N-(dibenz[b,f]oxepin-10-ylmethyl)-N-propyl-N-benzylamine;
N-allyl-N-benzyl-N-(dibenz[b,f]oxepin-10-ylmethyl)amine;
1-(dibenz[b,f]oxepin-10-ylmethyl)-4-methyl-piperazine;
1-(dibenz[b,f]oxepin-10-ylmethyl)-4-(2-hydroxyethyl)-piperazine;
N,N-diethyl-N-(dibenz[b,f]oxepin-10-ylmethyl)amine;
N-(dibenz[b,f]oxepin-10-ylmethyl)-N,N-dimethylamine;
N-(dibenz[b,f]oxepin-10-ylmethyl)-N-methylamine;
1-(dibenz[b,f]oxepin-10-ylmethyl)pyrrolidine;
N-[1-(dibenz[b,f]oxepin-10-ylethyl)-N,N-dimethylamine;
N-[1-(dibenz[b,f]oxepin-10-ylethyl)-N-methylamine;
1-(8-methoxy-dibenz[b,f]oxepin-10-ylmethyl)-4-methyl-piperazine;
N-(8-methoxy-dibenz[b,f]oxepin-10-ylmethyl)-N,N-dimethylamine;
N-(8-methoxy-dibenz[b,f]oxepin-10-ylmethyl)-N-methylamine;
N-(dibenz[b,f]oxepin-10-ylmethyl)amine;
N-butyl-N-(dibenz[b,f]oxepin-10-ylmethyl)amine;
N-(8-chloro-dibenz[b,f]oxepin-10-ylmethyl)-N,N-dimethylamine and
N-(8-chloro-dibenz[b,f]oxepin-10-ylmethyl)-N,N-diethylamine
   and of pharmaceutically acceptable salts thereof, in the manufacture of a medicament for the treatment of a degenerative ocular disorder, which is in particular selected from dry AMD, wet AMD and retinal detachment.

Another aspect pertains to a method of treating a degenerative ocular disorder in a patient In need of such treatment, comprising the administration of a therapeutically effective amount of a 10-aminoaliphatyl-dibenz[b,f]oxepine of formula I according to the above definition and/or above preferences to said patient.

The administration is preferably pertaining to oral, rectal, parenteral and topical administration. A even more preferred administration pertains to topical administration.

In general, this invention pertains also to an embodiment of any independent and dependant claim of the present application.

Efficacy in the described ocular disorders might be established for example In the following animal models:
1) Spontaneous development of a secondary form of glaucoma (e.g. pigment dispersion, angle closure or angle dysgenesis) in mice (for example, but not exclusively, strains DBA/2J, DBA/2Nnia, and AKXD28/Ty mice as described in Anderson et al., BMC Genetics 2001; 2:1, Chang et al., Nature Genetics 1999; 21: 405-409, John et al., Invest. Ophthalmol. Vis. Sci. 1998; 39: 951-962, Sheldon et al., Lab. Animal Sci. 1995; 15:508-518)
2) Genetic animal models for retinal degeneration, e.g. rd mouse (as described in Li et al., Invest. Ophthalmol. Vis. Sci. 2001; 42: 2981-2989), Rpe65-deficient mouse (Van Hooser et al., PNAS 2000; 97: 8623-8628), RCS rat (Faktorovich et al., Nature 1990; 347:83-86), rds mouse (Ali et al., Nature Genetics 2000, 25: 306-310), rcd1 dog (Suber et al., PNAS 1993; 90: 3968-3972)
3) Experimental retinal degeneration induced by

light exposure in mice (as described in Wenzel et al., Invest. Ophthalmol. Vis. Sci. 2001; 42: 1653-1659) or rats (Faktorovich et al., J. Neurosci: 1992; 12: 3554-3567)

administration of N-methyl-N-nitrosourea (Kiuchi et al., Exp. Eye Res. 2002; 74: 383-392) or sodium iodate (Sorsby & Harding, Vision Res. 1962; 2: 139-148).
4) Experimental model for the injury of the optic nerve (ON)

by ON crush in mice (Levkovitch-Verbin et al., Invest. Ophthalmol. Vis. Sci. 2000; 41: 4169-4174) and rats (Yoles and Schwartz, Exp. Neurol. 1998; 153:1-7)

by ON transection in rats (as described in Martin et al., Invest. Ophthalmol. Vis. Sci. 2002; 43:2236-2243, Solomon et al. J. Neurosci. Methods 1996; 70:21-25)

by experimental transient (acute) retinal ischemia in rats after ophthalmic vessel ligature (as described in Lafuente et al., Invest. Ophthalmol. Vis. Sci. 2001; 42:2074-2084) or cannulation of the anterior chamber (Buchi et al., Ophthalmologica 1991; 203:138-147)

by intraocular endothelin-1 injection in rats (Stokely at al., Invest. Ophthalmol. Vis. Sci. 2002; 43: 3223-3230) or rabbits (Takei et al., Graefes Arch. Clin. Exp. Ophthalmol 1993; 231:476-481)

The pharmaceutical compositions of this Invention comprise, for example, enteral or parenteral administration forms from approximately 10% to approximately 80%, preferably from approximately 20% to approximately 60%, active ingredient. Pharmaceutical compositions according to the invention for enteral or parenteral administration are, for example, in unit dose form, such as in the form of dragées, tablets, capsules or suppositories, and also ampoules. They are prepared in a manner known per se, for example by means of conventional mixing, granulating, confectioning, dissolving or lyophilising processes. For example, pharmaceutical compositions for oral administration can be obtained by combining the active ingredient with solid carriers, if desired granulating a resulting mixture, and processing the mixture or granules, if desired or necessary, after the addition of appropriate excipients, into tablets or dragée cores.

Suitable carriers are especially fillers, such as sugars, for example lactose, saccharose, mannitol or sorbitol, cellulose preparations and/or calcium phosphates, for example tricalcium phosphate or calcium hydrogen phosphate, and also binders, such as starch pastes using, for example, corm, wheat, rice or potato starch, gelatin, tragacanth, methylcellulose and/or polyvinylpyrrolidone, if desired disintegrators, such as the above-mentioned starches, also carboxymethyl starch, crosslinked polyvinylpyrrolidone, agar, alginic acid or a salt thereof, such as sodium alginate. Excipients are especially flow agents, flow conditioners and lubricants, for example silicic acid, talc, stearic acid or salts thereof, such as magnesium or calcium stearate, and/or polyethylene glycol. Dragée cores are provided with suitable, optionally enteric, coatings, there being used, inter alia, concentrated sugar solutions which may comprise gum arabic, talc, polyvinylpyrrolidone, polyethylene glycol and/or titanium dioxide, or coating solutions in suitable organic solvents or solvent mixtures, or, for the preparation of enteric coatings, solutions of suitable cellulose preparations, such as acetylcellulose phthalate or hydroxypropylmethylcellulose phthalate. Dyes or pigments may be added to the tablets or drage coatings, for example for identification purposes or to indicate different doses of active ingredient.

Other orally administrable pharmaceutical compositions are hard gelatin capsules and also soft, sealed capsules made of gelatin and a plasticiser, such as glycerol or sorbitol. The hard gelatin capsules may comprise the active ingredient in the form of granules, for example in admixture with fillers, such as lactose, binders, such as starches, and/or glidants, such as talc or magnesium stearate, and if desired with stabilisers. In soft capsules the active ingredient is preferably dissolved or suspended in suitable liquids, such as fatty oils, paraffin oil or liquid polyethylene glycols, it likewise being possible for stabilisers to be added.

Suitable rectally administrable pharmaceutical compositions are, for example, suppositories that consist of a combination of the active ingredient with a suppository base material. Suitable suppository base materials are, for example, natural or synthetic triglycerides, paraffin hydrocarbons, polyethylene glycols or higher alkanols. Gelatin rectal capsules that comprise a combination of the active ingredient with a base material may also be used. Suitable base materials Include, for example, liquid triglycerides, polyethylene glycols and paraffin hydrocarbons.

There are suitable for parenteral administration by infusion and/or injection especially aqueous solutions of an active ingredient in water-soluble form, for example in the form of a water-soluble salt, and also suspensions of the active ingredient, such as corresponding oily suspensions, there being used suitable lipophilic solvents or vehicles, such as fatty oils, for example sesame oil, or synthetic fatty acid esters, for example ethyl oleate or triglycerides, or aqueous suspensions that comprise viscosity-increasing substances, for example sodium carboxymethylcellulose, sorbitol and/or dextran, and optionally also stabilisers.

The compounds may also be administered topically in or around the eye, for example as eyedrops, ophthalmic suspensions or ointments, subconjunctival, peribulbar, retrobulbar or intravitreal injectons, possibly with the use of slow-release devices, such as conjunctival inserts, microspheres or other periocular or intraocular depot devices.

The dosage of the active ingredient depends on the species of warm-blooded animal, the age and the individual condition and also on the mode of administration. Normally the estimated approximate daily dose in the case of oral administration to a patient weighing approximately 75 kg is from approximately 10 mg to approximately 500 mg. In the case of topical administration, the approximate estimated daily dosage may vary from 0.001 to 10 mg, depending on the mode of administration.

The following Examples serve to illustrate the invention.

### EXAMPLE 1

Tablets each comprising 50 mg of N-(dibenz[b,f]oxepin-10-ylmethyl)-N-methyl-N-prop-2-ynylamine or a salt, for example the hydrochloride, thereof, may be prepared as follows:

| | | |
|---|---|---|
| active ingredient | 500.0 | g |
| lactose | 500.0 | g |
| potato starch | 352.0 | g |
| gelatin | 8.0 | g |
| talc | 60.0 | g |
| magnesium stearate | 10.0 | g |
| silicon dioxide (highly dispersed) | 20.0 | g |

| | |
|---|---|
| ethanol | q.s. |

The active ingredient is mixed with the lactose and 292 g of the potato starch, and the mixture is moistened with an ethanolic solution of the gelatin and granulated through a sieve. After drying, the remainder of the potato starch, the magnesium stearate, the talc and the silicon dioxide are mixed in and the mixture is compressed to form tablets each weighing 145.0 mg and comprising 50.0 mg of active Ingredient; if desired, the tablets may be provided with dividing notches for finer adaptation of the dose.

### EXAMPLE 2

A sterile-filtered aqueous gelatin solution containing 20% cyclodextrins as solubiliser, comprising as active ingredient 3 mg of N-(dibenz[b,f]oxepin-10-ylmethyl)-N-methyl-N-prop-2-ynylamine or of a salt, for example the hydrochloride, thereof, is so mixed, with heating, under aseptic conditions with a sterile gelatin solution comprising phenol as preservative that 1.0 ml of solution has the following composition:

| | | |
|---|---|---|
| active ingredient | 3 | mg |
| gelatin | 150.0 | mg |
| phenol | 4.7 | mg |
| dist. water with 20% cyclodextrins as solubiliser | 1.0 | ml |

### EXAMPLE 3

For the preparation of a sterile dry substance for injection, comprising 5 mg of N-(dibenz[b,f]oxepin-10-ylmethyl)-N-methyl-N-prop-2-ynylamine or of a salt, for example the hydrochloride, thereof, 5 mg of one of the compounds of formula I mentioned in the preceding Examples are dissolved as active ingredient in 1 ml of an aqueous solution containing 20 mg of mannitol and 20% cyclodextrins as solubiliser. The solution is sterile-filtered and, under aseptic conditions, filled into a 2 ml ampoule, deep-frozen and lyophilised. Before use, the lyophilisate is dissolved in 1 ml of distilled water or 1 ml of physiological saline. The solution is administered intramuscularly or intravenously. The formulation can also be filled into double-chamber disposable syringes.

### EXAMPLE 4

In a manner analogous to that described in previous examples, it is also possible to prepare pharmaceutical compositions comprising a different compound according to any one of the previous examples, or
1-(dibenz[b,f]oxepin-10-ylmethyl)-4-methyl-piperazine;
1-(dibenz[b,f]oxepin-10-ylmethyl)-4-(2-hydroxyethyl)-piperazine;
10-allylaminomethyldibenz[b,f]oxepine;
10-aminomethyldibenz[b,f]oxepine;
10-benzylamlnomethyldibenz[b,f]oxepine;
10-butylaminomethyldibenz[b,f]oxepine;
N,N-diethyl-N-(dibenz[b,f]oxepin-10-ylmethyl)amine;
N-(dibenz[b,f]oxepin-10-ylmethyl)-N,N-dimethylamine;
N-(dibenz[b,f]oxepin-10-ylmethyl)-N-methylamine;
1-(dibenz[b,f]oxepin-10-ylmethyl)pyrrolidine;
N-[1-(dibenz[b,f]oxepin-10-ylethyl)-N,N-dimethylamine;
N-[1-(dibenz[b,f]oxepin-10-ylethyl)-N-methylamine;
1-(8-methoxy-dibenz[b,f]oxepin-10-ylmethyl)-4-methyl-piperazine;
(8-methoxy-dibenz[b,f]oxepin-10-ylmethyl)-dimethylamine;
(8-methoxy-dibenz[b,f]oxepin-10-ylmethyl)-methylamine;
(8-chloro-dibenz[b,f]oxepin-10-ylmethyl)-dimethylamine;
(8-chloro-dibenz[b,f]oxepin-10-ylmethyl)-diethylamine
   or in each case a pharmaceutically acceptable salt thereof.

### EXAMPLE 5

The neuroprotective activity of N-(dibenz[b,f]oxepin-10-ylmethyl)-N-methyl-N-prop-2-ynylamine hydrogen maleate for retinal ganglion cells (RGCs) is demonstrated in animal models mimicking glaucomatous neurodegenerative processes.

In particular, in adult Sprague-Dawley rats, transient retinal ischemia induced by selective ligature of ophthalmic vessels for 60 minutes, causes a 30% RGC death seven days after ischemia. N-(dibenz[b,f]oxepin-10-ylmethyl)-N-methyl-N-prop-2-ynylamine hydrogen maleate, administration via intraperitoneal injection, significantly rescues RGCs (for example, a 45% rescue rate is observed at 0.005 mg/kg).

Similarly, 0.1 mg/kg N-(dibenz[b,f]oxepin-10-ylmethyl)-N-methyl-N-prop-2-ynylamine hydrogen maleate administered subcutaneously significantly reduces the loss of RGCs and the increase in apoptotic nuclei being assessed 18 days after crush of the rat optic nerve.

1-14. (canceled)

15. A method of treating a degenerative ocular disorder in a patient in need of such treatment, the method comprising administering a therapeutically effective amount of a compound of formula I 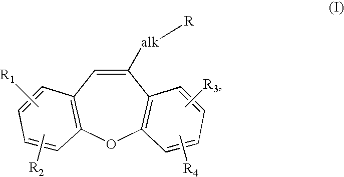
wherein alk is a divalent aliphatic radical,
R is an amino group which is unsubstituted or mono- or di-substituted by monovalent aliphatic and/or araliphatic radicals or disubstituted by divalent aliphatic radicals, and
R₁, R₂, R₃ and R₄ are each, independently of the others, hydrogen, lower alkyl, lower alkoxy, halogen or trifluoromethyl, and a pharmaceutically acceptable salt thereof.

16. The method of claim 15, wherein the degenerative ocular disorder is selected from the group consisting of ischemic retinopathies, anterior ischemic optic neuropathy, optic neuritis, age-related macular degeneration, diabetic retinopathy, cystoid macular edema, retinal detachment, retinitis pigmentosa, Stargardt's disease, Best's vitelliform retinal degeneration, Leber's congenital amaurosis and other hereditary retinal degenerations, pathologic myopia, retinopathy of prematurity, Leber's hereditary optic neuropathy, the after effects of corneal transplantation or of refractive corneal surgery and herpes keratitis.

17. The method of claim 16, wherein the degenerative ocular disorder is age-related macular degeneration.

18. The method of claim 17, wherein the age-related macular degeneration is dry age-related macular degeneration.

19. The method of claim 17, wherein the age-related macular degeneration is wet age-related macular degeneration.

20. The method of claim 16, wherein the degenerative ocular disorder is retinal detachment.

21. The method of claim 15, wherein
alk is methylene,
R is C₂-C₇alkenylamino, C₂-C₇alkynylamino, N—C₂-C₇alkenyl-N—C₁-C₄alkylamino, NC₂-C₇alkynyl-N—C₁-C₄alkylamine, pyrrolidino, piperidino or morpholino,
R₁ and R₃ are each, independently of the others, hydrogen, C₁-C₄alkyl, C₁-C₄alkoxy, halogen, or trifluoromethyl, and
R₂ and R₄ are hydrogen,

22. The method of claim 15, wherein
alk is methylene,
R is C₂-C₇alkenylamino, C₂-C₇alkynylamino, N—C₂-C₇alkenyl-N—C₁-C₄alkylamino, N—C₂-C₇alkynyl-N—C₁-C₄alkylamino, or phenyl-C₁-C₄alkylamino that is unsubstituted or substituted by C₁-C₄alkyl, C₁-C₄alkoxy, halogen, and/or by trifluoromethyl, and
R₁, R₂, R₃ and R₄ are hydrogen.

23. The method of claim 15, wherein the compound of formula I is selected from:
N-(dibenz[b,f]oxepin-10-ylmethyl)-N-methyl-N-prop-2-ynylamine;
N-allyl-N-(dibenz[b,f]oxepin-10-ylmethyl)amine;
N-allyl-N-(dibenz[b,f]oxepin-10-ylmethyl)-N-methylamine;
N-(dibenz[b,f]oxepin-10-ylmethyl)amine;
N-(dibenz[b,f]oxepin-10-ylmethyl)-N-prop-2-ynylamine;
N-(dibenz[b,f]oxepin-10-ylmethyl)-N-propylamine;
N-(dibenz[b,q]oxepin-10-ylmethyl)-N-methyl-N-propylamine;
1-dibenz[b,f]oxepin-10-ylmethyl-piperidine;
4-dibenz[b,f]oxepin-10-ylmethyl-morpholine;
N-(1-chloro-dibenz[b,f]oxepin-10-ylmethyl)-N-methyl-N-prop-2-ynylamine;
1-(1-chloro-dibenz[b,f]oxepin-10-ylmethyl)-pyrrolidine;
N-(1-chloro-dibenz[b,f]oxepin-10-ylmethyl)-N-methyl-N-propylamine;
N-methyl-N-prop-2-ynyl-N-(3-trifluoromethyl-dibenz[b,f]oxepin-10-ylmethyl)amine
1-(3-trifluoromethyl-dibenz[b,f]oxepin-10-ylmethyl)-pyrrolidine;
N-(7-chloro-dibenz[b,f]oxepin-10-ylmethyl)-N-methyl-N-prop-2-ynylamine;
1-(7-chloro-dibenz[b,f]oxepin-10-ylmethyl)-pyrrolidine;
N-(8-methoxy-dibenz[b,f]oxepin-10-ylmethyl)-N-methyl-N-prop-2-ynylamine;
N-(8-tert-butyl-dibenz[b,f]oxepin-10-ylmethyl)-N-methyl-N-prop-2-ynylamine;
1-(8-tert-butyl-dibenz[b,f]oxepin-10-ylmethyl)-pyrrolidine;
N-(6-chloro-dibenz[b,f]oxepin-10-ylmethyl)-N-methyl-N-prop-2-ynylamine;
1-(6-chloro-dibenz[b,f]oxepin-10-ylmethyl)pyrrolidine;
N-(1-fluoro-dibenz[b,f]oxepin-10-ylmethyl)-N-methyl-N-prop-2-ynylamine;
1-(1-fluoro-dibenz[b,f]oxepin-10-ylmethyl)pyrrolidine;
N-(dibenz[b,f]oxepin-10-ylmethyl)-N-benzylamine;
N-benzyl-N-(dibenz[b,f]oxepin-10-ylmethyl)-N-methylamine;
N-(dibenz[b,f]oxepin-10-ylmethyl)-N-propyl-N-benzylamine;
N-allyl-N-benzyl-N-(dibenz[b,f]oxepin-10-ylmethyl)amine;
1-(dibenz[b,f]oxepin-10-ylmethyl)-4-methyl-piperazine;
1-(dibenz[b,f]oxepin-10-ylmethyl)-4-(2-hydroxyethyl)-piperazine;
N,N-diethyl-N-(dibenz[b,f]oxepin-10-ylmethyl)amine;
N-(dibenz[b,f]oxepin-10-ylmethyl)-N,N-dimethylamine;
N-(dibenz[b,f]oxepin-10-ylmethyl)-N-methylamine;
1-(dibenz[b,f]oxepin-10-ylmethyl)pyrrolidine;
N-[1-(dibenz[b,f]oxepin-10-ylethyl)-N,N-dimethylamine;
N-[1-(dibenz[b,f]oxepin-10-ylethyl)-N-methylamine;
1-(8-methoxy-dibenz[b,f]oxepin-10-ylmethyl)-4-methyl-piperazine;
N-(8-methoxy-dibenz[b,f]oxepin-10-ylmethyl)-N,N-dimethylamine;
N-(8-methoxy-dibenz[b,f]oxepin-10-ylmethyl)-N-methylamine;
N-(dibenz[b,f]oxepin-10-ylmethyl)amine;
N-butyl-N-(dibenz[b,f]oxepin-10-ylmethyl)amine;
N-(8-chloro-dibenz[b,f]oxepin-10-ylmethyl)-N,N-dimethylamine and
N-(8-chloro-dibenz[b,f]oxepin-10-ylmethyl)-N,N-diethylamine
and of pharmaceutically acceptable salts thereof.

24. The method of claim 15, wherein the compound of formula I is selected from the group consisting of
N-(dibenz[b,f]oxepin-10-ylmethyl)-N-methyl-N-prop-2-ynylamine;
N-(dibenz[b,f]oxepin-10-ylmethyl)-N-prop-2-ynylamine;
N-(1-chloro-dibenz[b,f]oxepin-10-ylmethyl)-N-methyl-N-prop-2-ynylamine;
N-methyl-N-prop-2-ynyl-N-(3-trifluoromethyl-dibenz[b,f]oxepin-10-ylmethyl)amine
N-(7-chloro-dibenz[b,f]oxepin-10-ylmethyl)-N-methyl-N-prop-2-ynylamine;
N-(8-methoxy-dibenz[b,f]oxepin-10-ylmethyl)-N-methyl-N-prop-2-ynylamine;
N-(8-tert-butyl-dibenz[b,f]oxepin-10-ylmethyl)-N-methyl-N-prop-2-ynylamine;
N-(6-chloro-dibenz[b,f]oxepin-10-ylmethyl)-N-methyl-N-prop-2-ynylamine;
N-(1-fluoro-dibenz[b,f]oxepin-10-ylmethyl)-N-methyl-N-prop-2-ynylamine;
and of pharmaceutically acceptable salts thereof.

25. The method of claim 24, wherein the compound of formula I is N-(dibenz[b,f]oxepin-10-ylmethyl)-N-methyl-N-prop-2-ynylamine and/or a pharmaceutically acceptable salt thereof.

26. The method of claim 15, wherein the administration is selected from the group consisting of oral, rectal, parenteral and topical administration.
